(19) United States
(12) Patent Application Publication
(10) Pub. No.: US 2024/0382773 A1
(43) Pub. Date: Nov. 21, 2024

(54) IMPLANTABLE CRANIAL ELECTRONICS

(71) Applicant: University of Pittsburgh - Of the Commonwealth System of Higher Education, Pittsburgh, PA (US)

(72) Inventor: Joseph Peter Rubin, Pittsburgh, PA (US)

(21) Appl. No.: 18/694,219

(22) PCT Filed: Sep. 21, 2022
(86) PCT No.: PCT/US2022/044193

§ 371 Date: Mar. 21, 2024

Related U.S. Application Data
(60) Provisional application No. 63/246,921, filed on Sep. 22, 2021.

(51) Int. Cl. A61N 1/375; A61N 1/05; A61N 1/36; A61N 1/372; A61N 1/378

(52) U.S. Cl. CPC A61N 1/3758; A61N 1/0534; A61N 1/36125; A61N 1/36128; A61N 1/37211; A61N 1/3787

(57) ABSTRACT

A cranial electronics device includes electronics and a flexible body via which the electronics are carried. The flexible body and the electronics are formed such that the device has a lower surface approximating a contour of an outer surface of a portion of a patient's skull. The cranial electronics device is configured to be implanted between the patient's scalp and the portion of patient's skull.

### CROSS-REFERENCE TO RELATED APPLICATIONS

This application claims benefit of U.S. Provisional Patent Application Ser. No. 63/246,921, filed Sep. 22, 2021, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Computer-controlled treatment of various medical conditions has existed from some time. In some instances, a computerized controller is worn by the patient or may even be implanted. As, for example, illustrated in FIG. 1, in deep brain stimulation (DBS), an electronics housing 10 containing a controller may be implanted in subcutaneous tissue of the chest and lead wires 20 run across the neck of the patient and pass through the skull to form or to connect to one or more stimulating electrodes 30 implanted in the brain. The controller within electronics housing 10 sends electrical signals through wire electrodes 30 implanted in the brain.

DBS has revolutionized the treatment of complex neurological disorders such as Parkinson's disease, dystonia, and essential tremor. Newer applications of DBS are rapidly emerging and are being refined for conditions such as epilepsy, psychiatric disorders, and chronic pain. These expansions of the therapeutic use of DBS will result in an increasing number of patients and implanted electrodes. However, the widespread application of DBS will be accompanied by an increasing number of complications associated with the implanted hardware. Ultimately, a patient's relief of symptoms after DBS will be limited by hardware-related complications. Hardware failure can result from malfunction, lead migration, fracture, and infection. Scalp erosion, with exposure of underlying hardware, can lead to infection. Erosion of the overlying scalp occurs at points of increased tension between the skin and the hardware. The reported incidence for scalp erosion is between 1.4 and 8.3%. Factors that likely predispose a patient to scalp erosion include scalp thickness and the use of bulky hardware that has sharp edges. Measures to prevent scalp erosion are vital in decreasing morbidity for patients with implanted neurostimulation systems. One study found a 4.5% risk of hemorrhage associated with lead removal. In addition, the patient is exposed to all the known risks associated with reimplantation of a DBS system, including risks of sedation, infection, and intracranial hemorrhage.

The emerging field of brain-computer interface (BCI) technology has opened the possibility of enabling control of one's environment through thoughts. In that regard, humans can use the electrical signals from brain activity measured by one or more sensors (for example, an external sensor or an implanted electrode/electrode array) to interact with, influence, or change their environments. BCI technology may, for example, allow individuals unable to speak and/or use their limbs to once again communicate or operate assistive devices for walking and manipulating objects. Brain-computer interface research is an area of high public awareness and there is intense curiosity and interest in the field. It is hoped that one day soon BCI will dramatically improve the lives of many disabled persons affected by a number of different disease processes.

Brain-computer interfaces may, for example, acquire brain signals, analyze them, and translate them into commands that are relayed to output devices that carry out desired actions. BCIs do not use normal neuromuscular output (efferent) pathways. An important goal of BCI is to replace or restore useful function to people disabled by neuromuscular disorders such as amyotrophic lateral sclerosis, cerebral palsy, stroke, or spinal cord injury. From initial demonstrations of electroencephalography-based spelling and single-neuron-based device control, researchers have on to use electroencephalographic, intracortical, electrocorticographic, and other brain signals for increasingly complex control of cursors, robotic arms, prostheses, wheelchairs, and other devices.

Brain-computer interfaces may also prove useful for rehabilitation after stroke and for other disorders. In the future, BCIs might augment the performance of surgeons or other medical professionals. Moreover, brain-computer interface may also be used in treatment and/or diagnosis of a number of medical conditions.

Brain-computer interface technology is the focus of a rapidly growing research and development enterprise that is greatly exciting scientists, engineers, clinicians, and the public in general. The future uses of such technology may depend significantly on advances in a number of areas. For example, brain-computer interfaces require signal-acquisition hardware that is convenient, portable, safe, and able to function in all environments. Brain-computer interface systems also need to be validated in long-term studies of real-world use by, for example, people with severe disabilities. Effective and viable models for their widespread dissemination need to be implemented. Moreover, for certain uses, the day-to-day and moment-to-moment reliability of BCI performance must be improved so that it approaches the reliability of natural muscle-based function.

### SUMMARY

In one aspect, a cranial electronics device (sometime referred to herein as a device) includes electronics and a flexible body via which the electronics are carried. The flexible body and the electronics are formed (as an integrated, composite unit) such that the device has a lower surface approximating a contour of an outer surface of a portion of a patient's skull. The cranial electronics device is configured to be implanted between the patient's scalp and the portion of patient's skull. In a number of embodiments, the cranial electronics device has a maximum thickness of no greater than 20 mm, no greater than 10 mm, or no greater than 8 mm. An upper surface of the cranial electronics device may, for example, be formed to approximate the contour of the lower surface over a portion thereof other than a perimeter portion thereof. The cranial electronics device may, for example, be tapered in thickness in the perimeter portion thereof. The portion of the patient's skull may, for example, include or be a portion of the calvarium.

In a number of embodiments, data from a scan using an imaging system is used to determine the shape of the portion of the patient's skull. Such data may, for example, be determined from a computerized tomography scan.

In a number of embodiments, the cranial electronics device is sufficiently flexible to be folded during implantation. The flexible body may, for example, be formed from a biocompatible polymer (for example, a silicone, a polyurethane, or a perfluorinated polymer). In a number of embodiments, the flexible body includes polydimethylsiloxane.

In a number of embodiments, the thickness of the cranial electronics device does not vary more than 10% from an average thickness over the portion of the cranial electronics device other than the perimeter portion thereof.

In a number of embodiments, at least a portion of the electronics is flexible. At least a portion of the electronics may, for example, be housed within a hermetically sealed housing at least partially encompassed/embedded within or by the flexible body. The electronics may, for example, include a plurality of space nodes, wherein each of the plurality of nodes includes a hermetically sealed housing and includes a portion of (or one or more components of) the electronics positioned within the hermetically sealed housing. The plurality of nodes may, for example, be placed in electronic connection via flexible, electronic connectors.

The electronics may, for example, include a processor system, a memory system in operative connection with the processor system, a communication system in operative connection with the processor system, a sensor system in operative connection with the processor system, and a power system to provide energy/power to such systems. In a number of embodiments, the sensor system includes one or more sensors within the flexible body. The sensor system may additionally or alternatively include one or more sensors on the surface of the cranial electronics device. In a number of embodiments, the sensor system include an EEG sensor or an array of EEG sensors.

In a number of embodiments, the electronics is configured to be placed in operative connection with at least one other device implanted to interface with tissue via a wired connection or a wireless connection. The at least one other device may, for example, include an electrode configured to provide a stimulative electrical signal to the tissue. The at least one other device may, for example, include a sensor to detect a physiologic variable. In a number of embodiments, the sensor includes one or more electrodes which interface with neural tissue.

In a number of embodiments, the sensor is configured to acquire signals from the neural tissue and transmit the signals to the electronics, and the memory system includes software stored therein and executable by the processor system to process the signal. The processor system may, for example, analyze the signals, translate them to commands (or determine commands from such signals), and relay the commands to a remote output device via the communication system to carry out a defined action.

The cranial electronics device may further include a pharmaceutical delivery system in operative connection with the electronics. The pharmaceutical delivery system may be carried by or integrated with the flexible body.

In another aspect, a method of forming a cranial electronics device includes determining a contour of an outer surface of a portion of a patient's skull and forming the cranial electronics device to include a flexible body which carries electronics such that a lower surface of the cranial electronic device has a contour approximating a contour of the outer surface of a portion of a patient's skull. The cranial electronics device is further formed to be implanted between the patient's scalp and the portion of patient's skull. The (flexible) cranial electronics device may, for example, have a maximum thickness of no greater than 20 mm, no greater than 10 mm, or no greater than 8 mm. The portion of the patient's skull may, for example, include or be a portion of the calvarium.

In a number of embodiments, an upper surface of the cranial electronics device is formed to approximate the contour of the lower surface of the cranial electronics device over a portion thereof other than a perimeter portion thereof, and the cranial electronics device is tapered in thickness in the perimeter portion thereof. The thickness of the cranial electronics device may, for example, not vary more than 10% from an average thickness over the portion of the cranial electronics device other than the perimeter portion thereof.

In a number of embodiments, data from a scan using an imaging system is used to determine the contour of the outer portion of the patient's skull and, therefrom, to determine the contour of the lower surface of the flexible cranial electronics device. Such data may for example, be determined from a computerized tomography scan. As set forth above, the portion of the patient's skull may be a portion of the calvarium.

In a number of embodiments, the cranial electronics device is sufficiently flexible to be folded during implantation. The flexible body may, for example, be formed from a biocompatible polymer (for example, a silicone, a polyurethane, or a perfluorinated polymer). In a number of embodiments, the flexible body comprises polydimethylsiloxane.

In a number of embodiments, at least a portion of the electronics is flexible. At least a portion of the electronics may, for example, be housed within a hermetically sealed housing at least partially encompassed/embedded within or by the flexible body. The electronics may, for example, include a plurality of spaced nodes, wherein each of the plurality of nodes includes a hermetically sealed housing and a portion of the electronics positioned within the hermetically sealed housing. The plurality of nodes may, for example, be placed in electronic connection via flexible, electronic connectors.

In a number of embodiments, the electronics includes a processor system, a memory system in operative connection with the processor system, a communication system in operative connection with the processor system, a sensor system in operative connection with the processor system, and a power system to provide power to such systems. The sensor system may, for example, include one or more sensors within the flexible body. The sensor system may additionally or alternatively include one or more sensors on the surface of the cranial electronics device. In a number of embodiments, the sensor system include an EEG sensor or an array of EEG sensors.

In a number of embodiments, the electronics is configured to be placed in operative connection with at least one other device implanted to interface with tissue via a wired connection or a wireless connection. The at least one other device may, for example, include an electrode configured to provide a stimulative electrical signal to the tissue. The at least one other device may, for example, include a sensor to detect a physiologic variable. Such a sensor may, for example, include one or more electrodes which interface with neural tissue.

One or more sensors may, for example, be configured to acquire signals from the neural tissue and transmit the signals to the electronics. The memory system may, for example, include software stored therein and executable by the processor system to process the signal. The processor system may, for example, analyze the signals, translate the signals to commands, and relay the commands to a remote output device via the communication system to carry out a defined action.

As described above, the cranial electronics device may further include a pharmaceutical delivery system in operative connection with the electronics. The pharmaceutical delivery system may be carried by or integrated with the flexible body.

In another aspect, a method of implanting electronics in a patient includes forming a cranial electronics device including a flexible body carrying electronics, the cranial electronics device including a lower surface having a contour approximating a contour of an outer surface of a portion of patient's skull, the cranial electronics device being configured to be implanted between the patient's scalp and the portion of the patient's skull, and implanting the cranial electronics device between the patient's scalp and the portion of the patient's skull.

In a further aspect, an electronics device includes electronics and a flexible body via which the electronics are carried. The flexible body and the electronics may, for example, be formed (as an integrated, composite unit) such that the device has at least one surface approximating a contour of a surface of the internal body adjacent which the electronic device is to be implanted.

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates a currently available embodiment of a deep brain stimulation system.

FIG. 2A illustrates schematically, in an idealized cutaway view, a representative positioning relative to the skull/brain of an embodiment of a cranial electronics device or system hereof implanted in a patient.

FIG. 2B illustrates schematically an embodiment of a cranial electronics device or system hereof.

FIG. 2C illustrates a side, cross-sectional view of an embodiment of a cranial electronics device hereof.

FIG. 2D illustrates schematically another embodiment of a cranial electronics device or system hereof in which electronic components are housed in individual sealed housing connected by flexible electrical connectors.

FIG. 3A illustrates an anterosuperior view of embodiment of a cranial electronics device hereof implanted below a patient's scalp, between the scalp and outer surface of the calvarial bone.

FIG. 3B illustrates a lateral view of the cranial electronics device of FIG. 3A implanted below the patient's scalp.

FIG. 3C illustrates an implantation of the cranial electronics device of FIG. 3A through an incision in the scalp wherein the cranial electronics system is folded over on itself or rolled on itself to enable implantation via a relatively small incision.

FIG. 4A illustrates a right side view of an embodiment of a flexible body for a cranial electronics device hereof on a model of a skull of a patient designed via computed tomography (CT) scanning of the patient's skull.

FIG. 4B illustrates a top view of the flexible body of FIG. 4A on the model of the skull of a patient.

FIG. 4C illustrates a left side view of the flexible body of FIG. 4A on the model of the skull of a patient.

FIG. 4D illustrates a back view of the flexible body of FIG. 4A on the model of the skull of a patient.

FIG. 4E illustrates a front view of the flexible body of FIG. 4A on the model of the skull of a patient.

FIG. 4F illustrates a top isometric view of the flexible body of FIG. 4A on the model of the skull of a patient.

FIG. 4G illustrates a right side transparent view of the flexible body of FIG. 4A on the model of the skull of a patient.

FIG. 5A is a photograph illustrating a perspective view of another embodiment of a cranial electronics device hereof.

FIG. 5B is a photograph illustrating a perspective view of the cranial electronics system of FIG. 5A hereof positioned upon over the calvarial bone of a model of a skull.

FIG. 6 illustrates a top plan view of an embodiment of a cranial electronics device hereof next to a standard DBS electronics housing box and a cell phone for size comparison.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “an electrode” includes a plurality of such electrodes and equivalents thereof known to those skilled in the art, and so forth, and reference to “the electrode” is a reference to one or more such electrodes and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

The terms “electronics”, “electronic circuitry”, “circuitry” or “circuit,” as used herein include, but are not limited to, hardware, firmware, software, or combinations of each to perform a function(s) or an action(s). For example, based on a desired feature or need, a circuit may include a software-controlled microprocessor, discrete logic such as an application specific integrated circuit (ASIC), or other programmed logic device. A circuit may also be fully embodied as software. As used herein, “circuit” is considered synonymous with “logic.” The term “logic”, as used herein includes, but is not limited to, hardware, firmware, software, or combinations of each to perform a function(s) or an action(s), or to cause a function or action from another component. For example, based on a desired application or need, logic may include a software-controlled microprocessor, discrete logic such as an application specific integrated circuit (ASIC), or other programmed logic device. Logic may also be fully embodied as software.

The term “processor,” as used herein includes, but is not limited to, one or more of virtually any number of processor systems or stand-alone processors, such as microprocessors, microcontrollers, central processing units (CPUs), and digital signal processors (DSPs), in any combination. The processor may be associated with various other circuits that support operation of the processor, such as random-access memory (RAM), read-only memory (ROM), programmable read-only memory (PROM), erasable programmable read only memory (EPROM), clocks, decoders, memory controllers, or interrupt controllers, etc. These support circuits may be internal or external to the processor or its associated electronic packaging. The support circuits are in operative communication with the processor. The support circuits are not necessarily shown separate from the processor in block diagrams or other drawings.

The term “controller,” as used herein includes, but is not limited to, any circuit or device that coordinates and controls the operation of one or more input and/or output devices. A controller may, for example, include a device having one or more processors, microprocessors, or central processing units capable of being programmed to perform functions.

The term “software,” as used herein includes, but is not limited to, one or more computer readable or executable instructions that cause a computer or other electronic device to perform functions, actions, or behave in a desired manner. The instructions may be embodied in various forms such as routines, algorithms, modules, or programs including separate applications or code from dynamically linked libraries. Software may also be implemented in various forms such as a stand-alone program, a function call, a servlet, an applet, instructions stored in a memory, part of an operating system or other type of executable instructions. It will be appreciated by one of ordinary skill in the art that the form of software is dependent on, for example, requirements of a desired application, the environment it runs on, or the desires of a designer/programmer or the like.

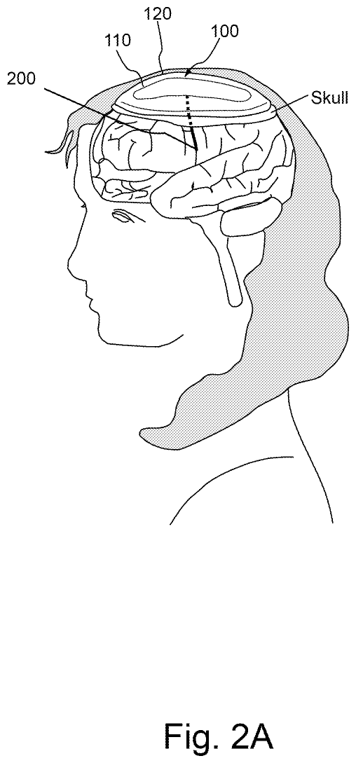

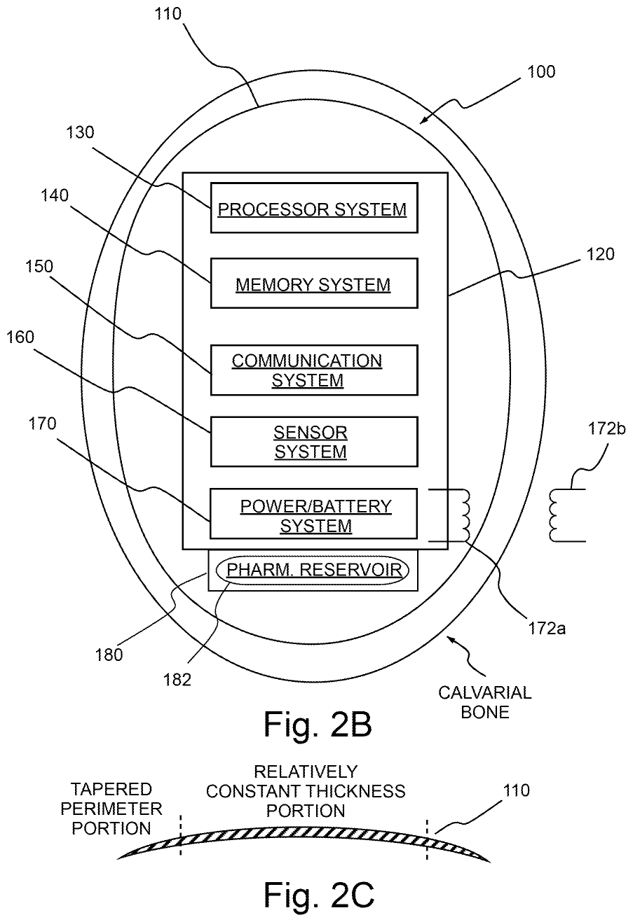

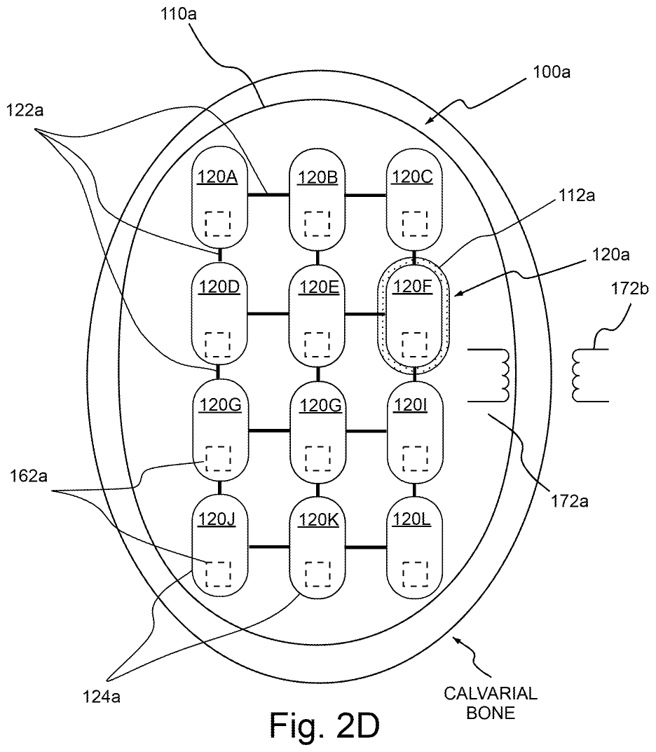

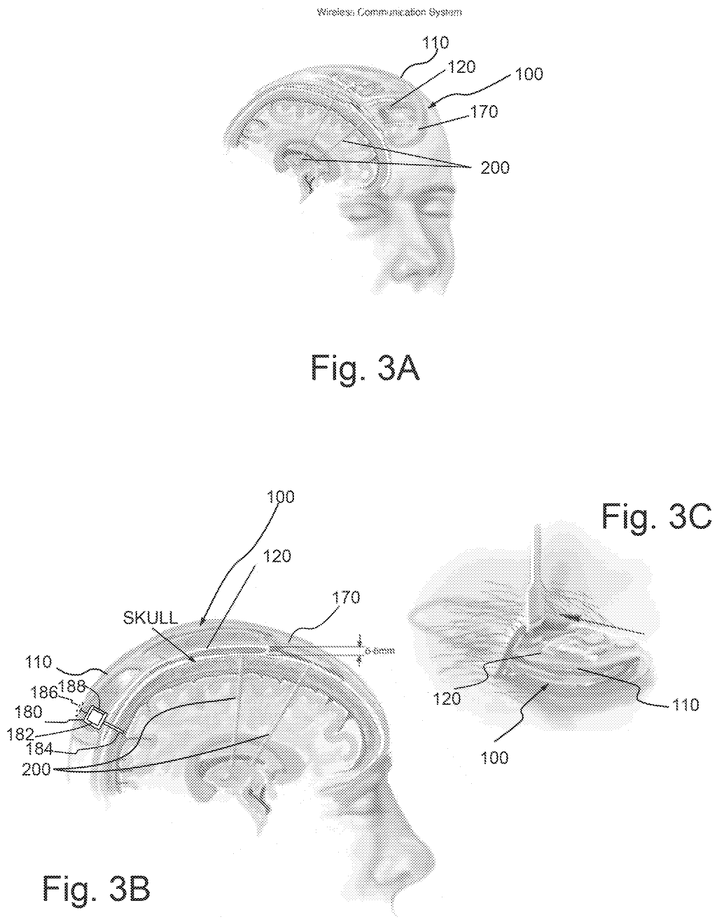

As, for example, illustrated in FIGS. 2A through 3C, in a number of embodiments hereof, a cranial (or cranially implantable) electronics device or system 100 includes a flexible body 110 to carry electronics 120. The flexile body 110 (or carrier) may, for example, be formed (for example, molded) from a flexible and biocompatible polymeric material such as a polyurethane, a perfluorinated polymer (for example, polytetrafluoroethylene), a silicone etc. In a number of embodiments, the biocompatible polymer is an elastomer. In a number of embodiments, the polymeric material is a silicone (or polysiloxane) elastomer such as polydimethylsiloxane (PDMS). Flexible body 110 may, for example, be formed of the same or a similar material as silicone implants currently approved for human implantation by the US Food and Drug Administration (FDA) and used throughout the body (including breast implants, implants for correction of pectus excavatum, orbital floor implants, external ear implants, and a host of other implants used in reconstructive surgery). As, for example, illustrated in FIGS. 2C, 3A and 3B, cranial electronics device 100 (that is, the integrated or composite structure including flexible body 110 and electronics 120) is formed to have a lower surface which is shaped or contoured to conform to an outer or upper contour of a portion of an individual patient's skull or the periosteum over the patient's skull (for example, extending over at least a portion of the calvarial bone). The calvaria is the top part of the skull and includes the upper portion of the neurocranium, forming the main component of the skull roof. The calvaria includes the superior portions of the frontal bone, the occipital bone, and the parietal bones. In a number of embodiments, the lower surface of cranial electronics device 100 is contoured to the shape of the periosteum/skull such that at least 50% (or at least 75%) of the area of the lower surface is in contact with the periosteum/skull when device 100 is implanted.

As illustrated in FIG. 3C, the thickness and flexibility of flexible body 110 (and nature/arrangement of associated electronics 120) may be such that device 100 may be folded (for example, folded or rolled over itself) to enable implantation via a relatively small incision (for example, extending between 20 to 50% of the width of device 100 in an unfolded/unfurled state) and subsequently unfolded, unrolled, or unfurled after implantation. In a relaxed or unfolded state, device 100 may, for example, cover most of the calvarium, which is an avascular plane. Because of the large surface area of the calvarium, more electronic circuitry and more extensive power sources can be placed into the implant than can be placed in currently available devices that are implantable.

In a number of embodiments, the area of device 100 is at least 20 cm², at least 50 cm², at least 100 cm², at least 150 cm², or at least 200 cm². Device 100 may, for example, have a thickness of 20 mm or less. In a number of embodiments, device 100 has a thickness of 10 mm or less, a thickness between 5-10 mm, or a thickness between 6-8 mm. To help ensure sealing of electronics 120 in a number of embodiments, the thickness of electronics 120 may be limited so that a sealing layer of the material of flexible body 110 separates electronics 120 from bodily fluids when device 100 is implanted. In a number of embodiments, the thickness of electronics 120 (which may vary within flexible body 110/over the area of deice 100) may, for example, be limited to provide at least 1 mm of the material of flexible body 110 separating electronics 120 and bodily fluids. As described below, in embodiments in which at least a portion of the electronics is, for example, hermetically or otherwise sealed, flexible body 110 need not completely encompass that portion of the electronics. In embodiments in which conductive elements (wires, electrodes, etc.) pass through flexible body 110 to connect with electronics 120 seals or sealing elements may be provided as known in the electrical arts to ensure a sealed environment within flexible body 110.

For long term implantation, in a number of embodiments hereof, electronics 120a may, for example, include one or more individual, hermetically or otherwise sealed nodes as illustrated in FIG. 2D (nodes 120A through 120L in the illustrated embodiment). In that regard, for some electronics/electronic components, flexible body 110a may not provide a suitable vapor barrier over an extended term of implantation. In the case of a plurality of such nodes (for example, nodes 120A through 120L), the spaced nodes may, for example, be placed in electronic and/or communicative connection or networked via flexible electrical connections 122a (such as wires, ribbons, or similar connections/connectors as known in the electrical and computer arts) as illustrated in FIG. 2D. Each of spaced nodes 120A through 120L may, for example, include a housing 124a (formed, for example, from a biocompatible material such as a ceramic material, a metal material (for example titanium), a polymeric material, and/or other similar materials as known in the medical arts) to create a hermetically sealed environment therewithin. Spacing between/positions of nodes 120A through 120L can be readily determined on a case-by-case basis to allow folding of device 100a for implantation as illustrated in FIG. 3C or in other folding arrangements.

As described above, sealed nodes 120A through 120L and/or other sealed electronics need not be fully encompassed by or embedded within flexible body 110 but may be partially encompassed thereby or embedded therein to form device 100, 100a as an integrated composite of flexible body 110, 110a and electronics 120, 120a. For example, a portion of the surface of one or more of nodes 120A through 120L may form a portion for the surface of device 100a and form part of the contour of that surface as described above. Flexible body 110a thus operates as a carrier for integrated electronics 120a wherein various portions of electronic 120a may be fully embedded/encompassed within or partially embedded/encompassed within/by flexible body 110a.

Portions of electronics 120a such as one or more of nodes 120A through 120L may also, for example, be partially or fully embedded within/by flexible body 110 to provide some degree of movement or cushioning when acted upon by an outside force. For example, as illustrated in FIG. 2D, node 120F is partially of fully encompassed by a layer 112a which may be less stiff (for example, have a lower modulus of elasticity) than the remainder/surrounding portion of the material of flexible body 110a to provide a shock-absorbing or cushioning functionality. Layer 112a may, for example, be formed from a material different from the surrounding portion of flexible body 110a (for example, formed from a foamed material) or the material of flexible body 110 may have a different morphology in the region of node 120F (for example, a cellular or strutted morphology) defining layer 112a to provide shock absorbing or cushioning functionality in the region of node 120F.

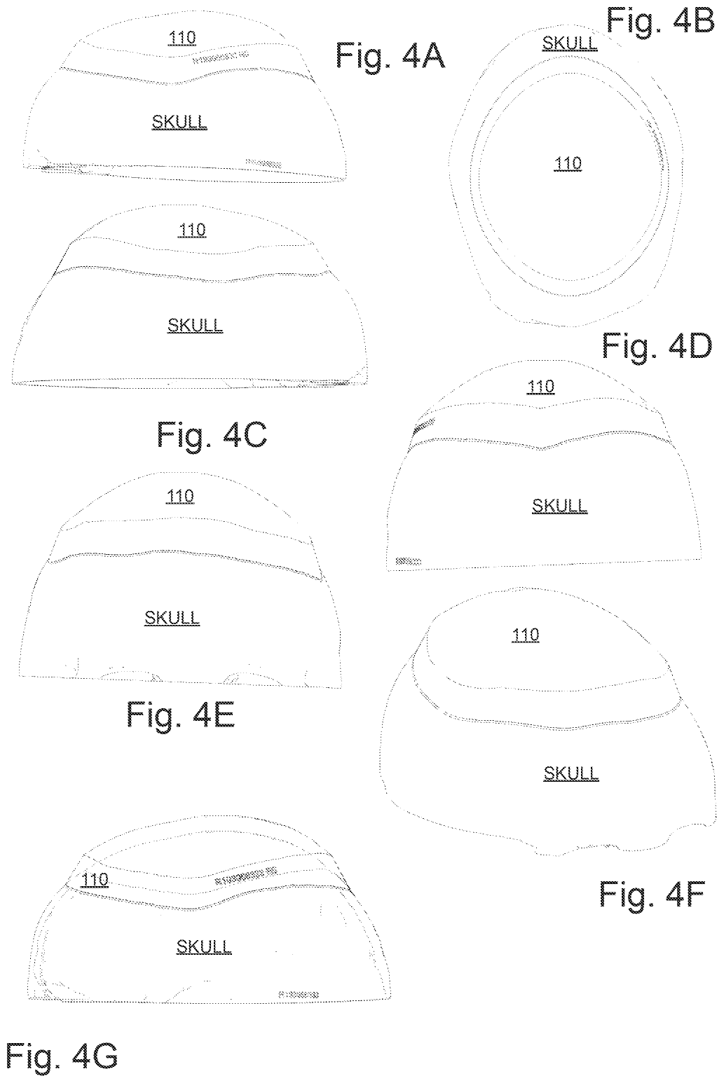

Flexible body 110, 110a of device 100, 100a (as well as electronics 120, 120a) may, for example, be custom designed for each patient by first determining the contour of the patient's scalp. That contour may, for example, be determined using an imaging system such as a computed tomography or CT system to scan the patient. Once the contour of the patient's outer table of the calvarium shape is determined, the lower or under surface of device 100, 100a (that is, the integrated, composite structure including flexible body 110, 110a and electronics 120, 120a) may be contoured/molded to fairly exacting standards to match the contour of the calvarium. Likewise, the upper or outer surface of device 100, 100a may be shaped to represent the patient's natural skull contour by, for example, ensuring that the thickness of device 100, 100a does not vary significantly over at least a portion of the area thereof. By matching the contour of the calvarium and maintaining a relatively low profile, device 100, 100a will be essentially undetectable when in place. A representative embodiment of a flexible body 110 for use in a device 100 hereof was modeled via CT scans and is illustrated in FIGS. 4A through 4G. Flexible body 110 of FIG. 4A through 4G is illustrates positioned on a model of the calvarium/skull of the scanned patient. After modeling, both flexible body 110 and the model skull were 3D printed. As, for example, illustrated in FIG. 2B and in FIGS. 4A through 4G, the edges or a perimeter portion of flexible body 110 may be tapered in thickness to varying degrees (which may be determined on a per-patient basis) to assist in creating a natural appearance after implantation.

Electronics 120 or 120a may, for example, be at least partially molded into polymeric (for example, silicone) flexible body 110, 110a as described above. The use of biocompatible polymers such as silicone in flexible body 110, 110a enables relatively good sealing of electronics/electronic circuitry 120, 120a, high biocompatibility, and an implant that resists tissue ingrowth so the device 100, 100a can be easily removed if desired or if necessary. As discussed above, hermetically-sealed nodes such as nodes 120A through 120L may be used to provide improved sealing over extended periods of use. Moreover, such independently sealed electronic components may form a part of a surface of device 100, 100a.

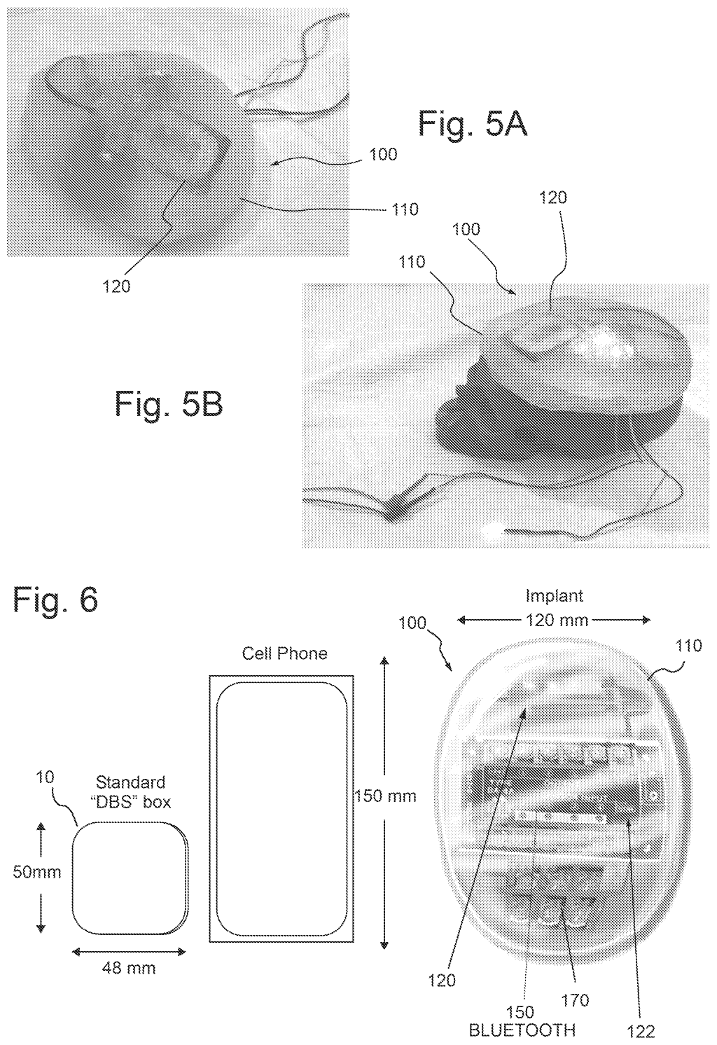

FIGS. 5A and 5B illustrate another embodiment of device 100 hereof which was molded from silicone. In FIG. 5B, device 100 is positioned over a model skull. In the embodiment of FIGS. 5A and 5B, embedded electronics 120 can be used to sense signals through wires that extend from the implant to the neural tissues (and/or via wireless communication with one or more electrodes in operative connection with neural tissues) and/or to transmit a signal to the nervous system.

As described above, because of the large surface area of the calvarium, more electronic circuitry and more extensive battery power sources can be placed into the implant than can be placed in devices that are implanted in smaller devices/housings. Flexible and relatively thin printed circuit boards may be used to support various electronic devices such as microprocessors, memory system, sensor systems, and communication systems. Moreover, a plurality of relatively small devices (for example, circuit boards, processors, memory components, batteries, communication components, etc.), at least some of which may be present within individual nodes including hermetically-sealed housings as described above in connection with FIG. 2D, may be connected by flexible conductors within flexible body 110. Flexible body 110 can, for example, be used to at least partially encompass and seal any type of electronic circuitry. Electronics or electronic circuitry 120 may, for example, include a processor system 130 (for example, including one or more microprocessors) and a memory system 140 in communicative connection with processor system 130. A communication system 150 may be provided in operative connection with one or more components of electronics 120 (such as processor system 130) to provide wired and/or wireless communication to devices or systems external to flexible body 110. A sensor system 160 may, for example, provide a wired and/or wireless interface for communication with external sensors. Sensor system 160 may also or alternatively include one or more sensors encompassed within flexible body 110. One or more sensors (or sensor arrays) within flexible body 110 and/or upon flexible body 110/device 100 may, for example, be used to detect electrical activity within neural tissues (for example, encephalographic sensors for detecting brain signals). In a number of embodiments a sensor or sensor array is positioned in or on flexible body 110 to measure electroencephalographic (EEG) signals (that is, brainwave signals or electrical activity in the cerebral cortex). Continuous EEG monitoring may, for example, occur. An array of sensors 162a is represented schematically in FIG. 2D in dashed lines as being positioned on the bottom of housings 124a. Such an array or a portion thereof can, alternatively, be attached to the lower surface of device 100, 100a. A power system 170 (which may, for example, include power storage devices such as batteries and/or capacitors) may, for example, be included to provide power to one or more other electronics components of device 100. Power system 170 may be readily separable from the remainder of device 100 (for example, via sealable contact elements) so that power system 170 may be more easily be removed/replaced. Power system 170 may also be positioned to be near the original implantation incision to facilitate removal. Moreover, power system 170 may be inductively rechargeable (to, for example, recharge one or more batteries and/or capacitors) via an implanted coil 172a and an external coil 172b (see, FIGS. 2B and 2D) as known in the transcutaneous energy transfer arts.

Although cranial electronics device 100, 100a may be used in connection with any system or methodology in which it is desirable to implant electronics within a patient for therapeutic and/or diagnostic purposes, device 100, 100a is very useful for providing an electronic interface to organ tissue and particularly to components of the nervous system (for example, neural tissue) accessible from the head. For example, device 100, 100a can include or be placed in communicative connection (either in a wired or a wireless manner) with an interface device placed into contact with an organ (for example, the brain). Such interface devices include, but are not limited to, sensors/electrodes which may be used to detect a physiological variable (for example, a neural electrical signal, EEG, blood flow, temperature, pressure, pH, etc.) and/or to transmit an electrical signal into tissue. In a number of embodiments, such interface devices may, for example, include one or more conductive elements or wires or an array of electrically conductive elements.

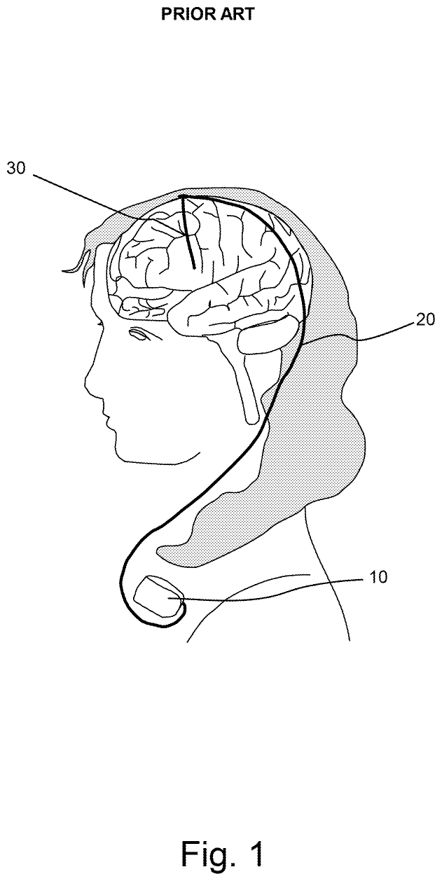

As described above, the use of deep brain stimulation or DBS has revolutionized the treatment of a number of complex neurological disorders. In a number of embodiments, device 100 hereof provides significant improvements in DBS. FIGS. 3A and 3B illustrate device 100 hereof in electrical (wired) connection of device 100 with DBS electrodes 200. Both efferent (active stimulations) and afferent (sensing) wires or leads can be provided. As illustrates in FIGS. 2A, 3A and 3B, device 100 conforms to the skull shape on both the proximal (skull) and distal (scalp) surfaces. The upper surface of device 100 may, for example, significantly recreate the shape of the skull to be imperceptible to an observer even in the case of bald patients. As described above, the undersurface of device 100 may be custom shaped using, for example, CT scanning to conform essentially exactly to the patient's skull. Once again, a low-profiled body, which may include tapered edges, will blend well into the skull shape, evenly distributing pressure and holding much more electronics than a standard DBS box while being better hidden and eliminating wiring which crosses the neck as illustrated in FIG. 1 for currently available systems.

FIG. 6 illustrates the significantly greater surface area and volume provided by an embodiment of device 100 compared to a standard DBS electronics box/housing 10. In the illustrated embodiment, a portion of electronics 120 (including a communication system 150 configured for BLUETOOTH wireless communication) is in operative connection with a flexible printed circuit board 122. Power/battery system 170 includes a flexible battery A described above, standard DBS electronic housings 10 are currently implanted in the chest, and wires are run under the skin of the neck to the scalp and then into the brain through holes drilled into the skull. Such wires can erode through the scalp because of focal pressure. Moreover, the wires can break in the neck. In the case of device 100, the implant and circuitry are much closer to the end of the lead wires/electrodes, providing a more accurate signal because the distance is greatly reduced. Moreover, device 100 also solves the problem of excessive wear and breakage of the wire resulting from motion of the neck. Implantation of device 100 in the scalp will be much better tolerated than the implant of an electronics housing in the chest. As described above, device 100 may, for example, have a very low profile (for example, approximately 5-10, 5-8 or 6-8 millimeters in vertical height) and be relatively large in area so that the pressure is evenly distributed under the scalp. Device 100 may thereby eliminate the problem of wires eroding through the scalp because of focal pressure.

The global deep brain stimulation devices market size was valued at USD 1.12 billion in 2020 and is expected to expand at a compound annual growth rate (CAGR) of 9.3% from 2021 to 2028. Increasing numbers of patients suffering from involuntary movements associated with Parkinson's disease, dystonia, and multiple sclerosis is expected to drive market growth. In addition, there is an increasing demand for minimally invasive techniques as a result of enhanced patient outcomes. Deep brain stimulation, on average, costs about $30,000 plus physician and MRI fees. The surgery, when recommended by a doctor, is typically covered by insurance. Device 100, 100a may, for example, provide a less expensive, more efficient, and less invasive alternative to current technologies, while significantly reducing or eliminating patient complications arising from hardware device of currently available DBS systems.

Device 100, 100a may also, for example, be used in brain-computer interface or BCI applications. Once again, device 100, 100a may be used in transmission of signals to and/or detection of signals from the brain. In a number of embodiments, device 100, 100a may provide the following functions via execution via processor system 130 of software stored in memory system 140 of implantable electronics 120 thereof: (1) signal acquisition, (2) feature extraction, (3) feature translation, and (4) device output. As known in the BCI arts, device 100, 100a may be used in, for example, advancing efforts in visions restoration and the control of powered prosthetic and/or devices. Device 100, 100a provides the ability to implant larger and more powerful computer and/or other electronic components in close vicinity to the brain, thus opening BCI technology to a larger market span. Currently, in BCI system which obtain signals from one or more implanted sensor/electrodes, percutaneous wiring is required to transmit such signals to an extracorporeal computer system for processing, significantly limiting the technology. As the technology for localized neural interfaces evolves, every more powerful microprocessors or arrays thereof can be implanted via device 100, 100a. Moreover, communications with systems external to device 100, 100a (including devices to be controlled as well as implanted sensors) can be achieved with a wireless communication protocol such as the BLUETOOTH protocol via communication system 150. Wireless transmission to/from device 100, 100a may be used both in detecting of signals via implanted sensors/electrodes and in control of remote device functions.

As further illustrated in FIGS. 2B and 3B, device 100 or other device hereof may include a pharmaceutical/drug delivery system 180 in operative connection with and controllable by electronics 120 for delivery of one or more pharmaceutical compositions (for example, a chemotherapy drug, an antiseizure drug, etc.) from one or more reservoirs 182 via an implanted conduit 184 (see FIG. 3B). Pharmaceutical/drug reservoir(s) 182 may, for example, be fillable via one or more percutaneous ports 186 as illustrated in FIG. 3B. A liquid pharmaceutical composition within reservoir 182 may, for example, be pressurized using a small pump 188 (illustrated schematically in FIG. 3B) as known in the medical arts (for example, as used in insulin delivery pumps). Pharmaceutical/drug delivery system 180 may, for example, be at least partially housed in a titanium housing as described above.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A cranial electronics device, comprising:
electronics, and
a flexible body via which the electronics are carried,
the flexible body and the electronics being formed such that the cranial electronics device comprises a lower surface having a contour approximating a contour of an outer surface of a portion of a patient's skull, the cranial electronics device being configured to be implanted between the patient's scalp and the portion of the patient's skull.

2. The cranial electronics device of claim 1 wherein the cranial electronics device has a maximum thickness of no greater than 20 mm.

3. (canceled)

4. (canceled)

5. The cranial electronics device of claim 1 wherein an upper surface of the cranial electronics device is formed to approximate the contour of the lower surface of the cranial electronics device over a portion thereof other than a perimeter portion thereof, and the cranial electronics device is tapered in thickness in the perimeter portion thereof.

6. (canceled)

7. The cranial electronics device of claim 1 wherein data from a computerized tomography scan is used to determine the contour of the outer portion of the patient's skull.

8. The cranial electronics device of claim 1 wherein the portion of the patient's skull is a portion of the calvarium.

9. The cranial electronics device of claim 1 wherein the cranial electronics device is sufficiently flexible to be folded during implantation.

10. The cranial electronics device of claim 1 wherein the flexible body is formed from a biocompatible polymer.

11. The cranial electronics device of claim 10 wherein the flexible body comprises a silicone, a polyurethane, or a perfluorinated polymer.

12. (canceled)

13. The cranial electronics device of claim 5 wherein the thickness of the cranial electronics device does not vary more than 10% from an average thickness over the portion of the cranial electronics device other than the perimeter portion thereof.

14. (canceled)

15. The cranial electronics device of claim 1 wherein at least a portion of the electronics is housed within a sealed housing at least partially encompassed by the flexible body.

16. The cranial electronics device of claim 1 wherein the electronics comprises a plurality of spaced nodes, each of the plurality of spaced nodes comprising a sealed housing, and a portion of the electronics within the sealed housing.

17. The cranial electronics device of claim 16 wherein the plurality of spaced nodes are placed in electronic connection via flexible, electronic connectors.

18. The cranial electronics device of claim 1 wherein the electronics comprises a processor system, a memory system in operative connection with the processor system, a communication system in operative connection with the processor system, a sensor system in operative connection with the processor system, and a power system.

19. The cranial electronics device of claim 18 wherein the sensor system comprises one or more sensors within the flexible body.

20. The cranial electronics device of claim 18 wherein the sensor system comprises one or more sensors on the upper surface or the lower surface of the cranial electronics device.

21. The cranial electronics device of claim 18 wherein the sensor system comprises an array of EEG sensors.

22. The cranial electronics device of claim 1 wherein at least a portion of the electronics is flexible and the electronics is configured to be placed in operative connection with at least one other device implanted to interface with tissue via a wired connection or a wireless connection.

23. The cranial electronics device of claim 22 wherein the at least one other device comprises an electrode configured to provide a stimulative electrical signal to the tissue.

24. The cranial electronics device of claim 22 wherein the at least one other device comprises a sensor to detect a physiologic variable.

25. The cranial electronics device of claim 24 wherein the sensor comprises one or more electrodes which interface with neural tissue.

26. The cranial electronics device of claim 25 wherein the sensor is configured to acquire signals from the neural tissue and transmit the signals to the electronics, and the memory system includes software stored therein and executable by the processor system to process the signal.

27. The cranial electronics device of claim 26 wherein the processor system analyzes the signals, translates them to commands, and relays the commands to a remote output device via the communication system to carry out a defined action.

28. The cranial electronics device of claim 1 further comprising a pharmaceutical delivery system in operative connection with the electronics.

29. The cranial electronics device of claim 18 further comprising a pharmaceutical delivery system in operative connection with the electronics.

30. A method of forming a cranial electronics device, comprising:
determining a contour of an outer surface of a portion of a patient's skull, and
forming the cranial electronic device to comprise a flexible body which carries electronics such that a lower surface of the cranial electronics device has a contour approximating a contour of an outer surface of a portion of a patient's skull, the cranial electronics device being formed to be implanted between the patient's scalp and the portion of the patient's skull.

31.-57. (canceled)

58. A method of implanting electronics in a patient, comprising:
forming a cranial electronics device comprising a flexible body carrying electronics, the cranial electronics device comprising a lower surface having a contour approximating a contour of an outer surface of a portion of patient's skull, the cranial electronics device being configured to be implanted between the patient's scalp and the portion of the patient's skull, and
implanting the cranial electronics device between the patient's scalp and the portion of the patient's skull.

59. (canceled)
